(19) United States
(12) Patent Application Publication
(10) Pub. No.: US 2023/0255563 A1
(43) Pub. Date: Aug. 17, 2023

(54) INSERTABLE PATIENT TABLE FOR PEDIATRIC OR SMALL PATIENTS FOR A MAGNETIC RESONANCE IMAGING SCANNER

(71) Applicants: Siemens Healthcare GmbH, Erlangen (DE); Research Institute at Nationwide Children's Hospital, Columbus, OH (US)

(72) Inventors: Arne Hengerer, Möhrendorf (DE); Heiko Rohdjess, Grossenseebach (DE); Aimee K. Armstrong, Columbus, OH (US)

(73) Assignees: Siemens Healthcare GmbH, Erlangen (DE); Research Institute at Nationwide Children's Hospital, Columbus, OH (US)

(21) Appl. No.: 17/672,992

(22) Filed: Feb. 16, 2022

(51) Int. Cl. A61B 5/00; A61B 5/055; G01R 33/341

(52) U.S. Cl. CPC A61B 5/704; A61B 5/055; G01R 33/341; A61B 2503/045

(57) ABSTRACT

The invention relates to an insertable patient table for a Magnetic Resonance Imaging scanner, wherein the patient table has a length of up to ± 20 % of the length of the bore of the main magnet of the scanner and is adapted for pediatric and/or small patients, and wherein the patient table is adapted to be inserted into the bore of the main magnet without having a stand standing on the floor of an examination room. The invention further relates to a Magnetic Resonance Imaging scanner comprising an insertable patient table and a method for positioning a small and/or pediatric patient within the bore of a main magnet of a Magnetic Resonance Imaging scanner.

The invention relates to an insertable patient table for a Magnetic Resonance Imaging Scanner, a Magnetic Resonance Imaging scanner comprising an insertable table and a method for positioning a small and/or pediatric patient within the bore of a main magnet of a Magnetic Resonance Imaging scanner.

Magnetic Resonance Imaging (MRI) is a medical imaging technique used for diagnosis of diseases and for medical interventions. A MRI scanner uses strong magnetic fields, magnetic field gradients and radiofrequency (RF) pulses to generate high-contrast cross-sectional images of the organs and/or physiological processes in the body. The MRI scanner itself usually resembles a tube with a bore, comprising a large cylindrical main magnet, gradient coils and radio-frequency coils. Usually, the MRI scanner further comprises a patient table having a stand on the floor of an examination room. Before carrying out the MRI examination, the patient is positioned on top of the patient table outside of the bore. Then, the table with the patient lying on top is moved into the bore, where the MRI examination is performed.

MRI interventions can be carried out as continuous in-bore interventions during the MRI examination. Alternatively, the MRI intervention can be carried out as a discontinuous intervention, comprising one or several interruptions of the MRI examination to move the patient out of the bore in order to perform the medical intervention. Then, the patient is moved back into the bore to continue the MRI examination. The dimensions of the slidable patient table are adapted to carry an adult person and to be moved into the bore. As the length of the bore is usually about 1.4 ±0.2 m, while the length of the patient table is about 2 m, a part of the patient table as known from the prior art protrudes from the bore.

During a medical intervention, physicians need to have access to the patient and to the respective medical equipment, which is in contact with the patient, such as catheters or needles. However, the access to the patient and the medical equipment in the bore is limited. To reach the intervention position, the physicians have to bend over the patient table or even bend into the scanner to reach the patient and/or the medical equipment. As the patient table obstructs the access to the bore, the access can be difficult, in particular for small physicians. In addition, the access to the patient and the evacuation of the patient from the bore can be time-consuming. This can have serious consequences for the patient in case of an emergency, such as a heart attack or a stroke, which requires fast evacuation from the bore and immediate treatment by the physician.

It is an object of the invention to provide MRI equipment which enables a facilitated access to the patient, in particular during continuous in-bore MRI interventions. A further object of the invention is to provide the MRI equipment, which allows facilitated discontinuous MRI interventions, and a fast evacuation of the patient and the patient table.

Said objects are met by an insertable patient table as claimed in claim 1, by a Magnetic Resonance Imaging scanner comprising an insertable patient table as claimed in claim 10 and a method for positioning a small and/or pediatric patient within the bore of a main magnet of a Magnetic Resonance Imaging scanner as claimed in claim 12.

According to one aspect of the present invention, the insertable patient table for a Magnetic Resonance Imaging scanner is adapted for a pediatric and/or small patient and has a length of up to ± 20 % of the length of the bore, preferably up to ± 10 % of the length of the bore, and most preferred up to the length of the bore of the main magnet. In an embodiment, the insertable patient table has a length between 70% and 100% of the length of the bore. The insertable patient table according to the invention is used instead of a patient table for adults as known from the prior art. The patient table according to the invention is adapted to be inserted into the bore of the main magnet without having a stand standing on the floor of an examination room of the MRI scanner. Due to the shorter length of the patient table and the absence of a stand standing on the floor, the access to the patient and/or medical equipment is facilitated since the physician can sit or stand directly outside the bore, at a position where in the prior art, the stand and/or part of the patient table would be. Further advantages of the invention are facilitated in-bore MRI interventions, and a fast evacuation of the patient and the patient table.

The main magnet is understood to mean a magnet which is adapted to generate a strong magnetic field (B₀-field) which can be used for the MRI examination. The main magnet may be of cylindrical shape. The bore is understood to mean the “hollow region” within the main magnet, where a homogeneous magnetic field is generated, and which includes the imaging volume around the isocenter. During MR imaging, the body part to be imaged is generally inside the bore. The patient table may be positioned below the isocenter so that the body part to be imaged is in the isocenter. In particular, the upper surface of the patient table may be positioned at a suitable height below the isocenter, which is typically at the center of the bore, so that isocenter is within the Region of Interest (ROI) which is to be examined by MRI. The upper surface or patient support of the patient table may e.g. be located 2-18 cm, preferably 5-15 cm, below the isocenter, when the patient table is positioned inside the bore.

The patient table may comprise an insert like a table, a board, a support or any other object on which a patient can be placed. A stand has to be understood to be any object, which stands on the floor of the room, where the MRI scanner is located and on which the patient table can be placed. According to the invention, the insertable patient table does not have a stand which would obstruct the access to the patient. The absence of a stand has the advantage of facilitated access to the patient.

The term “insertable” may mean that the patient table may be manually positioned, e.g. carried into the bore. However, embodiments where the movement of the patient table in and out of the bore is aided by a sliding mechanism and/or a motor are also encompassed by the invention. In most embodiments, however, at least a part of the patient table is adapted to be manually transferred into and out of the bore.

It is advantageous to adapt the dimensions of the patient table to the patient size. In an embodiment, the length of the patient table equals or exceeds the body height of the patient and is for example at least 3 cm longer than the body height of the patient. According to the invention, the length of the patient table can be up to the length of the bore ± 20 %. In terms of absolute length, the patient table may be shorter than 160 cm, preferably shorter than 140 cm, more preferred shorter than 120 cm and most preferred shorter than 100 cm. Therefore, the patient table does not protrude, or protrudes only up to 20 % of the bore length, from the bore. This has the advantage of facilitated access to the patient. The width of the patient table has to be smaller than the inner diameter of the bore, e.g. 40 to 80 cm, preferably 50 to 70 cm. For patients with different body heights, patient tables of different sizes can be used.

The term “patient” is to be understood to comprise humans, pets and animals. A pediatric patient can be a newborn, a toddler or an adolescent. A small patient can be a small human, pet or animal. A small patient may e.g. have a body height corresponding to the length of the patient table. For example, a small and/or pediatric patient may have a body height of up to 150 cm, preferably up to 130 cm and more preferred up to 100 cm. A small patient size enables the use of small patient tables. The patient table is e.g. adapted for patients having a weight up to 40 kg, preferably up to 30 kg, more preferred up to 25 kg. This has the advantage that the patient can be lifted manually onto the patient table, and the patient table can be manually transferred into the bore.

In an embodiment, the insertable patient table does not comprise a rail or rollers or an electric motor for sliding in and out of the bore, and can be inserted manually into the bore. This has the advantage that the insertable patient table can be inserted quickly into the MRI scanner. In another embodiment, the insertable table has a rail or rollers for sliding in and out of the bore, but no electric motor.

In most embodiments, the patient table of the invention is detached from the MR system, and its presence within the bore is not required in order to initiate an imaging procedure. This is different to most prior art systems, which require that a patient table be docked to the MRI scanner in order to start the MRI examination. In some embodiments of the invention, such a requirement can be met by docking an RF local coil to the MRI scanner, when using the insertable patient table according to the invention.

In a preferred embodiment, the insertable patient table comprises a base portion having a shape adapted to the lower half of the magnet bore, and a table top overlay adapted to be placed on top of the base portion and adapted to accommodate the patient during a Magnetic Resonance Imaging Examination. In particular, the lower contour of the base portion may be adapted or match the inner contour of the bore. The base portion can have a roughly semi-circular or semielliptic cross-section, wherein the flat side facing up may be located at or below the isocenter, so that the isocenter will be within the body part to be imaged. In particular, the base portion can be shaped as a cylinder segment being slightly smaller than a half cylinder and having a flat side facing up as a patient support, so that the patient itself, and in particular the ROI, may be centered within the magnet bore. The outer dimensions of the half cylinder may be smaller than the inner dimensions of the lower half of the magnet bore to enable the base portion to be moved into the bore. The length of the base portion can be up to the length of the bore ± 20%, preferably up to the length of the bore ± 10% and most preferred up to the length of the bore. Therefore, the base portion does not protrude, or protrudes only by up to 20% of the length of the bore, from the bore. This has the advantage of facilitated access to the patient.

The table top overlay can be understood to be a board, a support or any other object on which a patient can be placed.

The two-part form (base portion and table top overlay) of the patient table has the advantage that the patient may be very quickly evacuated from the bore by sliding the table top overlay from the base portion, leaving the base portion still in the bore. Therefore, the base portion may have a flat upper side, and the table top overlay is adapted for sliding on this upper side. In some embodiments, there may be a sliding mechanism such as a rail on which the table top overlay is guided e.g. along the z-direction on the base portion. In other embodiments, table top overlay can be moved freely on the flat upper side of the base portion. For example, the table top overlay may have ball rollers on its lower side, or the base portion may have ball rollers on its upper side. The free movability has the advantage that the table top overlay does not have to be oriented along the z-direction, but can be freely angulated. This enables more flexibility for MR imaging, and also allowing better access to the patient during and intervention. The two-part form of the patient table is also advantageous during discontinuous interventions, when the table top overlay has to be moved in and out of the bore several times.

In order to allow free placement of the table top overlay on the upper side of the base portion, the width of the table top overlay may be smaller than the width of the upper side of the base portion, e.g. by at least 10%, preferably by at least 20% and most preferred by 30% or more. Further, the length of the table top overlay may be smaller than the length of the base portion, e.g. by at least 10%, preferably by at least 20% and most preferred by 30% or more. The upper side of the table top overlay may include a layer of soft foam or fabric for comfortable placement of the patient.

In a further embodiment of the invention, the insertable patient table is adapted to accommodate a local radio-frequency (RF) coil for carrying out MRI examinations. Therefore, the RF coil is close to the patient and the position of the RF coil is moved together with the patient. This has the advantage of an improved image quality.

In an embodiment, the table top overlay has a non-adherent surface on the bottom and/or the base portion has a non-adherent upper surface. The non-adherent surface can be realized by coating with a non-adherent material, for example a synthetic material like plastic or Teflon®. This has the advantage that sliding of the table top overlay on the upper surface of the base portion is facilitated. Alternatively, the insertable patient table may comprise a device which is adapted to generate an air cushion on the bottom side of the table top overlay. A blower can be used as a device to generate an air cushion. Air, artificial air or nitrogen can be used as blowing gases. This has the advantage of facilitated sliding of the table top overlay on the upper surface of the base portion.

In a preferred embodiment, the table top overlay has grips for handling. The grips can be recesses or cut-outs which allow for easy gripping of the table top overlay. The grips can also be handles protruding from the table top overlay. The handles can be of the same material as the table top overlay or of a different material. The table top overlay can have one grip or several grips, e.g. one or two at each small end. The grips offer the advantage that the table top overlay, which accommodates the patient, can be manually placed on the base portion in any position, and the position can be easily altered during an intervention. In addition, the grip(s) allow to evacuate the patient fast in case of an emergency. There may be grips also in the base portion and the table top overlay, but in particular in the table top overlay, as this may be moved during an intervention. Also in embodiments without table top overlay, the patient table may have grips for easy carrying and handling.

According to a further embodiment, the patient table or a part of the patient table, in particular the table top overlay and/or the base portion, can be made of a rigid and/or light weight material. The patient table and the base portion can be made of several different materials, of which one or several materials are light weight and/or rigid. In particular, a middle layer or core of the patient table or of the base portion can be made of a rigid, light weight material. The light weight material can have a density of 40 to 300 kg/m³. The light weight material can be a rigid or nonrigid foam or a foamed plexiglass, for example Rohacell®, or a fiber-reinforced plastic or a combination of these materials. In some embodiments, the base portion is made of such light weight material, with a hard outer layer on the top face, on which the table top overlay is placed.

According to an embodiment, the patient table or a part of the patient table, in particular the base portion, can be a light weight construction. The light weight construction may comprise one or several loadbearing elements made of light materials, such as polymers or fiber-reinforced plastics. The light weight construction may comprise several elements fitted together to give the patient table support and shape. In other words, the light weight construction may not be solid, but may include cavities or voids or may be hollow, so that it does not completely fill out the lower half of the bore. The cavities may be formed by a lattice structure of loadbearing elements, or by a honeycomb structure of the light materials. The light weight construction can comprise one or several beams and/or boards which together form the patient table and can bear the weight of the patient. The light weight constructions may be formed from boards, panels or beams, for example as a truss. For example, a curved board having an outer contour matching the inner contour of the bore may be combined with a flat board forming the patient support placed on top of the curved board, thereby forming a substantially hollow construction. Further beams or panels may be disposed between the curved lower board and the flat upper board as reinforcement. The front and back ends of this construction may remain open. For example, the lightweight construction may comprise 2 to 10 beams, panels or boards. The lightweight construction can be supported by an inner lining of the bore, which may comprise rails, supports, holes or hooks.

According to an embodiment, the patient table or a part thereof is height-adjustable. This has the advantage that the height of the patient support or patient reclining area can be adapted to the size of the patient, to ensure that the ROI is within the isocenter. This can be realized by providing one or several boards which can be placed on top of the base portion, for example a set of boards or table top overlays of different heights. In another embodiment, several boards can be placed on top of each other, and possibly a table top overlay on top of the boards. In a further embodiment, the patient table is height-adjustable by adjusting the height of a table top overlay. This may be realized by a lead screw or a pneumatic mechanism.

According to an embodiment, the length of the patient table or a part thereof is adjustable. This has the advantage that the length can be adapted to the size of the patient, to ensure that the patient table can be handled as easily as possible. This can be realized by making the patient table or part thereof extendible, for example by a telescopic or drawer mechanism. In an advantageous embodiment, the extendible part is the table top overlay. It may comprise two pieces which are telescoped or interlocking, so that the length of the table top overlay may be adjusted. For example, it may be adjusted within a range of about 50 to 140 cm, preferably about 50 to 100 cm. This embodiment may require multiple sizes of mattresses to be placed on top of the patient table or table top overlay, but this may be less expensive for hospitals to buy than multiple table tops.

In a further embodiment, a local RF coil is incorporated into the table top overlay. Thus, the RF coil can be close to the patient and the position of the RF coiled can be moved together with the position of the patient. According to an embodiment, the table top overlay comprises a local RF coil which is adapted to be coupled inductively with a body coil. This has the advantage that no additional cables and/or plugs are needed and that the table top overlay can be used in various types of MRI scanners. In an alternative embodiment, the table top overlay does not contain a RF coil.

According to a further embodiment, the patient table and in particular the base portion comprises a recess adapted to hold a local RF coil. This cost-effective embodiment has the advantage that the RF coil can be inserted into the recess before or after the base portion has been inserted into the bore, but before the patient is slid into the imaging volume on the table top overlay.

In an embodiment, the upper surface of the table top overlay is smaller than the upper surface of the base portion. Therefore, the table top overlay can be at various angles to the z-direction of the main magnet. The z-direction is the direction of the main magnetic field and may be parallel to the floor and the upper surface of the base portion. A table top overlay, which is smaller than the upper surface of the base portion, has the advantage that the table top overlay, which accommodates the patient, can be rotated easily in order to position and/or reposition the patient inside the bore.

In an embodiment, the insertable patient table comprises a base portion and a set of table top overlays adapted to be placed on top of the base portion, and adapted to different patient sizes. In a set of table top overlays, each table top overlay can have a different length and/or width. A set of table top overlays may for example comprise one or more of the sizes 50±5 cm x 30±5 cm, 70±5 cm x 35±5 cm, 90±5 cm × 40±5 cm, 110±5 x 50±5 cm, 130±5 cm x 60±5 cm or 150±5 x 70±5 cm. Alternatively, a set can comprise table top overlays with different lengths, but having the same width, having the advantage that it fits different patient sizes and can be in particular suitable for short adult persons, pets or animals. The set can also be a combination of the before-mentioned sets. The set may also comprise table top overlays of different heights, to ensure that patients of different sizes may still be placed in the isocentre of the magnet. In particular, table top overlays of smaller length and width may be higher than those of larger length and width, as there are intended for smaller patients. The height may vary between 2 and 10 cm. The set can comprise between 2 and 20 different table top overlay sizes, preferably between 2 and 10 and more preferably between 3 and 6 different sizes.

According to an embodiment of the invention, the table top overlay is made of one or several non-conductive materials. This enables that resuscitative measures can be carried out while the patient is lying on the table top overlay. The patient can be quickly pulled from the bore together with the table top overlay in case of an emergency and does not have to be removed from the overlay before carrying out emergency measures, so that these can be carried out faster.

The features of the different embodiments can be combined with the features of the other embodiments. The advantages given for a feature also apply to the embodiments comprising this feature.

The invention is also directed to a Magnetic Resonance Imaging scanner comprising an insertable patient table as described herein. All advantages given for the insertable patient table also apply to the MRI scanner according to the invention.

The invention further is a method for positioning a small and/or pediatric patient within the bore of a main magnet of a Magnetic Resonance Imaging scanner. According to a preferred embodiment, the method comprises the following steps:
step S1: placing the base portion in the bore of the main magnet of the MRI scanner, wherein the base portion can stay in the bore for several interventions;
step S2: positioning the patient on the table top overlay outside of the bore, positioning the patient and/or the RF coil, optionally preparing the patient for the intervention e.g. by establishing venous access;
step S3: manually transferring the table top overlay with the patient to the bore;
step S4: adjusting the angle and position of the table top overlay on the upper surface of the base portion, optionally re-adjusting and re-positioning the table top overlay in particular to optimize access to the patient during the MRI examination.

The advantages given for the insertable patient table and MRI Scanner comprising the insertable patient table also apply for the method for positioning a small and/or pediatric patient. The features of the different embodiments can be combined with the features of the other embodiments. The advantages given for a feature also apply to the embodiments comprising this feature.

For example, a pediatric patient can be placed on a table top overlay, e.g. one suitable to its size, outside of the MR room. There, the patient is prepared for an intervention, e.g. by draping the covers, laying a port for venous access, positioning the RF local coil, e.g. a flex coil on or below the patient, and potentially giving an anaesthesia. In a next step, the table top overlay with the patient is manually carried to the MRI scanner and placed on top of the base portion. It is then possible to adjust the angle and position, as needed for the intervention. Advantageously, the physician may simply move the table top overlay manually by pulling and pushing on the upper side of the base portion, optionally using the handles provided on the table top overlay.

According to a further embodiment of the invention, the method for positioning a small and/or pediatric patient can further comprise one or both of the following steps:
step S5: carrying out the MRI examination;
step S6: removing the patient from the bore together with table top overlay, optionally removing the patient fast in case of an emergency.

This allows fast evacuation of the patient if needed. If handles or grips are provided on the table top overlay, these may be used for efficient and fast withdrawal of the table top overlay with the patient from the bore.

The individual steps do not necessarily have to be carried out sequentially. According to an embodiment, step S4 can be carried out several times before and/or during step S5. According to an embodiment, in particular for carrying out a discontinuous MRI examination, one or several or all of the steps S1 to S6 can be carried out more than once.

Further advantages and features of the invention will be apparent from the following description of preferred embodiments of the subject matter of the invention with reference to the accompanying drawings. The following description serves only for clarification of the invention and should not be understood as limiting the appended claims to any of the embodiments.

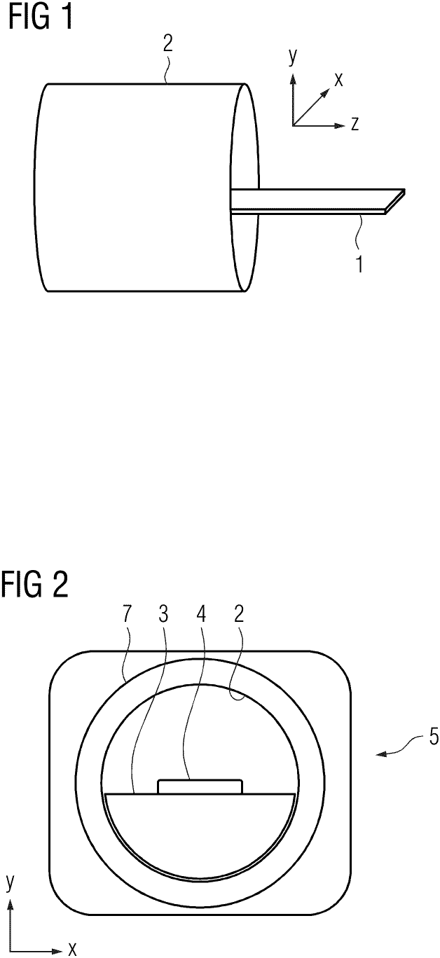

FIG. 1 shows a schematic side view of an insertable patient table according to an embodiment of the invention.

FIG. 2 shows a schematic front view of a MRI Scanner according to a first embodiment of the invention.

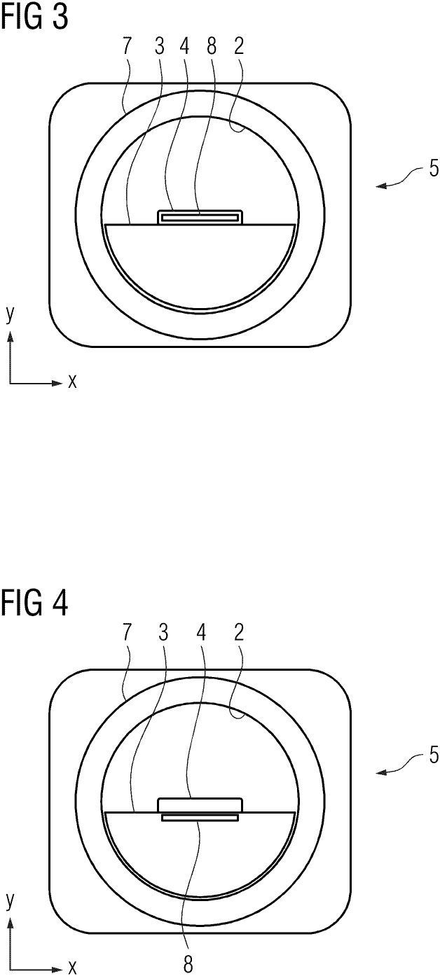

FIG. 3 shows a schematic front view of a MRI Scanner according to a second embodiment of the invention.

FIG. 4 shows a schematic front view of a MRI Scanner according to a third embodiment of the invention.

FIG. 5 shows a schematic top view of an insertable patient table according to a first embodiment of the invention.

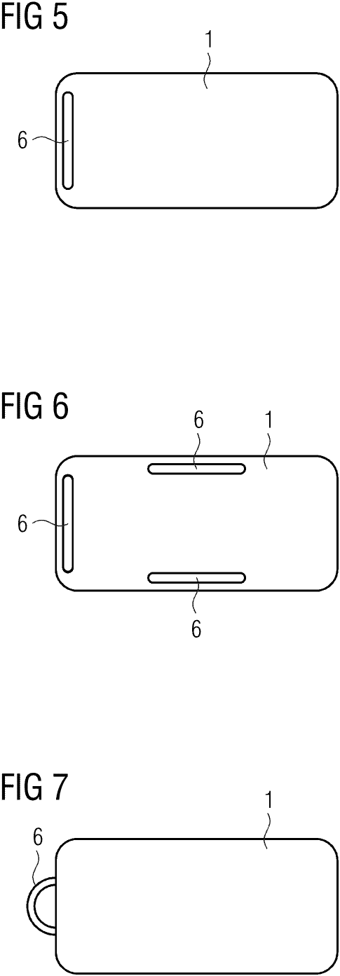

FIG. 6 shows a schematic top view of an insertable patient table according to a second embodiment of the invention.

FIG. 7 shows a schematic top view of an insertable patient table according to a third embodiment of the invention.

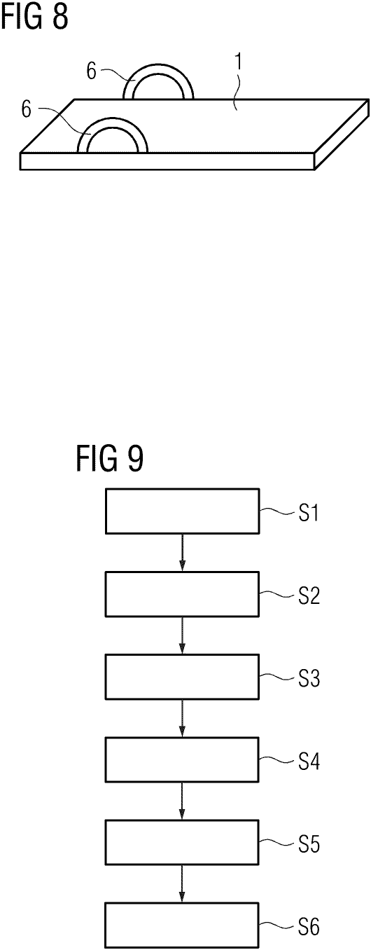

FIG. 8 shows a schematic top view of an insertable patient table according to a fourth embodiment of the invention.

FIG. 9 shows a flow chart illustrating a method according to an embodiment of the invention for positioning a small and/or pediatric patient within the bore of a main magnet of a Magnetic Resonance Imaging scanner.

FIG. 1 shows a schematic side view of an insertable patient table 1 according to an embodiment of the invention, which is adapted to be inserted into the bore 2 of a MRI scanner. The 2 bore has the shape of hollow cylinder oriented horizontally. The insertable patient table is adapted to be inserted into the bore along the z-direction. The insertable patient table does not have a stand standing on the floor of an examination room of the MRI scanner. The length of the insertable patient table is adapted to fit the size of a small and/or pediatric patient and is smaller or equal to the length of the bore, or longer by 20% at the most, so that the patient table according to the invention does not protrude from the bore by more than 20% of the bore length. Therefore, the access to the patient or the medical equipment is facilitated. Due to the absence of a stand the access is further facilitated and a fast evacuation of the patient in case of an emergency is enabled.

FIG. 2 shows a schematic front view of a MRI Scanner 5 according to a first embodiment of the invention, comprising a cylindrical main magnet 7, a bore 2, a base portion 3 and a table top overlay 4. The base portion 3 is shaped as a segment of cylinder and is placed within the bore such that the flat upper side is positioned at or a few centimeters below the central axis of the cylindrical bore, and the round side is on the bottom. The shape of the base portion is adapted to fit into the lower half, or slightly less than half, of the cylindrical bore. A table top overlay is placed on top of the base portion to enable moving the patient. The size of the table top overlay is adapted to fit into the bore. The size of the upper surface of the table top overlay is smaller than the upper surface of the base portion. The table top overlay is placed on top of the base portion, such that the longitudinal direction of the table top overlay may be at various angles to the z-direction of the main magnet and parallel to the upper surface of the base portion to enable positioning or repositioning the patient before and/or during MRI examination and intervention. In most cases, the distance of the top of the table overlay and the center of the bore is around half the body thickness of the patient (or width if the patient positioned on the side side).

FIG. 3 shows a schematic front view of a MRI Scanner 5 according to a second embodiment of the invention, comprising a cylindrical main magnet 7, a bore 2, a base portion 3 and a table top overlay 4, wherein the table top overlay comprises a RF coil 8. Therefore, the RF coil is close to the patient and the position of the RF coiled is moved together with the patient. This has the advantage of an improved image quality.

FIG. 4 shows a schematic front view of a MRI Scanner 5 according to a third embodiment of the invention, comprising a cylindrical main magnet 7, a bore 2, a base portion 3 and a table top overlay 4, wherein the base portion comprises a RF coil 8. This embodiment has the advantage of being inexpensive as no RF coil has to be inserted into every patient table of a set of patient tables.

FIG. 5 shows a schematic top view of an insertable patient table 1 according to an embodiment of the invention, comprising a grip in the shape of a recess 6. The recess 6 is located on the front side of the patient table 1. This enables handling the patient table 1 and facilitates moving and positioning the patient before and/or during the MRI examination. Further, adjusting the angle and position of the patient and/or the patient table 1 is facilitated.

FIG. 6 shows a schematic top view of an insertable patient table 1 according to an embodiment of the invention, comprising two recesses or cut-outs 6 for handling the patient table. According to an embodiment, the patient table 1 has two cut-outs 6. Each of the longer sides of the insertable patient table 1 has one cut-out 6. The presence of two cut-outs 6 has the advantage that there is a separate cut-out 6 for each hand, which facilitates handling the patient table 1.

FIG. 7 shows a schematic top view of an insertable patient table 1 according to an embodiment of the invention, comprising a grip 6 protruding from the surface of the patient table 1. The grip 6 is located at the front side of the insertable patient table 1 to facilitate handling the patient table 1.

FIG. 8 shows a schematic top view of an insertable patient table 1 according to an embodiment of the invention, comprising two handles 6, protruding from the surface of the patient table 1, to enable handling the patient table 1 with two hands.

The various recesses and grips shown in FIGS. 3 to 6 may be part of the table top overlay.

FIG. 9 shows a flow chart, illustrating a method for positioning a small and/or pediatric patient within the bore of a Magnetic Resonance Imaging scanner according to an embodiment of the invention. The method comprises the following steps:
step S1: placing the base portion in the bore of the main magnet of the MRI scanner;
step S2: positioning the patient on the table top overlay;
step S3: manually transferring the table top overlay with the pediatric patient to the bore;
step S4: adjusting the angle and position of the table top overlay on the upper surface of the base portion;
step S5: carrying out the MRI examination;
step S6: removing the patient from the bore by using the grips of the table top overlay.

1. Insertable patient table for a Magnetic Resonance Imaging scanner,
wherein the patient table has a length of up to ±20 % of the length of the bore of the main magnet of the scanner and is adapted for pediatric and/or small patients,
and wherein the patient table is adapted to be inserted into the bore of the main magnet without having a stand standing on the floor of an examination room of the Magnetic Resonance Imaging scanner.

2. Insertable patient table according to claim 1, comprising a base portion having a shape adapted to the lower half of the magnet bore, and a table top overlay adapted to be placed on top of the base portion and adapted to accommodate the patient during a Magnetic Resonance Imaging Examination.

3. Insertable patient table according to claim 1, which is adapted to accommodate a local RF coil for carrying out Magnetic Resonance Imaging examinations on the patient.

4. Insertable patient table according to claim 2, wherein the table top overlay has a non-adherent surface on the bottom in order to be slidable on the upper surface of the base portion.

5. Insertable patient table according to claim 2, comprising a device which is adapted to generate an air cushion on the bottom side of the table top overlay, such that sliding of the table top overlay on the upper surface of the base portion is facilitated.

6. Insertable patient table according to claim 2, wherein the table top overlay has one or several grips for handling.

7. Insertable patient table according to claim 2, wherein the patient table and in particular the base portion comprises a part made of a lightweight material, in particular a rigid foam.

8. Insertable patient table according to claim 1, wherein the patient table comprises a lightweight construction.

9. Insertable patient table according to claim 1, wherein the patient table or a part thereof is height-adjustable.

10. Insertable patient table according to claim 2, wherein a local RF coil is incorporated into the table top overlay.

11. Insertable patient table according to claim 1, wherein the length of the patient table or a part thereof, in particular the length of a table top overlay placed on top of a base portion, is adjustable.

12. Insertable patient table according to claim 2, wherein the patient table and in particular the base portion comprises a recess adapted to hold a local RF coil.

13. Insertable patient table according to claim 2, wherein the upper surface of the table top overlay is smaller than the upper surface of the base portion,
and wherein the table top overlay is adapted to be placed on top of the base portion, such that the longitudinal direction of the table top overlay may be at various angles to the z-direction of the main magnet and parallel to the upper surface of the base portion.

14. Insertable patient table according to claim 2, wherein the table top overlay is made of a non-conductive material.

15. Insertable patient table according to claim 2, comprising a base portion having a shape adapted to the lower half of the magnet bore, and a set of table top overlays adapted to be placed on top of the base portion, and adapted to a set of different patient sizes.

16. Magnetic Resonance Imaging scanner comprising an insertable patient table, wherein the patient table has a length of up to ±20% of the length of the bore of the main magnet of the scanner and is adapted for pediatric and/or small patients,
and wherein the patient table is adapted to be inserted into the bore of the main magnet without having a stand standing on the floor of an examination room of the Magnetic Resonance Imaging scanner.

17. Method for positioning a small and/or pediatric patient within the bore of a main magnet of a Magnetic Resonance Imaging scanner comprising an insertable patient table adapted to be inserted into the bore of the main magnet and having a length of up to ±20 % of the length of the bore of the main magnet, wherein the patient table comprises a base portion having a shape adapted to the lower half of the magnet bore, and a table top overlay adapted to be placed on top of the base portion and adapted to accommodate a patient during a Magnetic Resonance Imaging Examination,
The method comprising the following steps:
placing the base portion in the bore of the main magnet;
positioning the patient on the table top overlay;
manually transferring the table top overlay with the patient into the bore;
adjusting the angle and position or the table top overlay on an upper surface of the base portion, in particular to optimize patient access during the MRI examination.

18. Method for carrying out a MRI examination on a small and/or pediatric patient, comprising the positioning steps according to claim 17, and further comprising:
carrying out the MRI examination;
removing the patient from the bore using one or several grips.
